(19) United States
(12) Patent Application Publication
(10) Pub. No.: US 2006/0052375 A1
(43) Pub. Date: Mar. 9, 2006

(54) Bis-alkylbenzylamines

(76) Inventors: Sophie Marquais-Bienewald, Hegenheim (FR); Werner Holzl, Eschentzwiller (FR); Andrea Preuss, Basel (CH); Andreas Mehlin, Rheinfelden (DE); Frederic Brunner, Chezard (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/531,232

(22) PCT Filed: Oct. 14, 2003
(86) PCT No.: PCT/EP03/11384

§ 371 Date: Apr. 13, 2005

(30) Foreign Application Priority Data
Oct. 21, 2002 (EP) 02405898.4
Jan. 22, 2003 (CH) 0091/03

(51) Int. Cl. A61K 31/5377; A61K 31/4545; A61K 31/4178; C07D 413/02; C07D 401/02; C07D 403/02

(52) U.S. Cl. 514/231.8; 514/316; 514/397; 544/78; 546/186; 548/312.7; 514/649

(57) ABSTRACT

Compounds of Formula (1), wherein R₁ is hydrogen; C₁-C₁₈alkyl; trifluoromethyl; C₃-C₈cycloalkyl; phenylC₁-C₅alkyl; phenyl-C₁-C₅alkoxy; mono- or di-N-C₁-C₅alkamino-C₁-C₅alkyl; amino-di-N-C₁-C₅alkylamino-C₁-C₅alkyl; C₁-C₅alkoxy-C₁-C₅alkyl; R₂ is C₂-C₂₀alkyl; hydroxy-C₁-C₂₀alkyl; phenyl; phenyl-C₁₋C₅alkyl; phenyl-C₁-C₅alkoxy; mono- or di-N-C₁-C₅alkylamino-C₁-C₅alkyl; amino-di-N-C₁-C₅alkylamino-C₁-C₅alkyl; or heteroaryl-C₁-C₅alkyl; or R₁ and R₂ together with the nitrogen atom bonding them form a 5- to 7-membered monocyclic heterocyclic ring; are described. They are suitable for the antimicrobial treatment of surfaces, especially as antimicrobial active ingredients against gram-positive and gram-negative bacteria.

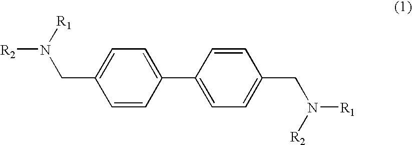

The present invention relates to bis-alkylbenzylamines, to the preparation of those compounds, and to their use in the antimicrobial treatment of surfaces, as antimicrobial active ingredients against gram-positive and gram-negative bacteria, yeasts and fungi, and in the preservation of cosmetics, household products, textiles, plastics, and for use in disinfectants.

The bis-alkylbenzylamines according to the invention correspond to formula 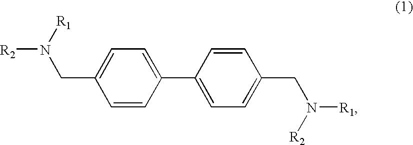
wherein
R₁ is hydrogen; C₁-C₁₈alkyl; trifluoromethyl; C₃-C₈cycloalkyl; phenyl-C₁-C₅alkyl; phenyl-C₁-C₅alkoxy; mono- or di-N-C₁-C₅alkylamino-C₁-C₅alkyl; amino-mono- or di-N-C₁-C₅alkylamino-C₁-C₅alkyl; C₁-C₅alkoxy-C₁-C₅alkyl;
R₂ is C₂-C₂₀alkyl; hydroxy-C₁-C₂₀alkyl; phenyl; phenyl-C₁-C₅alkyl; phenyl-C₁-C₅alkoxy; mono- or di-N-C₁-C₅alkylamino-C₁-C₅alkyl; amino-mono- or di-N-C₁-C₅alkylamino-C₁-C₅alkyl; or heteroaryl-C₁-C₅alkyl; or
R₁ and R₂ together with the nitrogen atom bonding them form a 5- to 7-membered mono-cyclic heterocyclic ring;
   with the proviso that compounds of formula (1) are excluded wherein
a. R₁ is hydrogen; and
   R₂ is butyl;
b. R₁ is hydrogen; and
   R₂ is cyclohexyl;
c. R₁ and R₂ are butyl;
d. R₁ and R₂ are propyl;
e. R₁ and R₂ together form a monocyclic ring of the formula 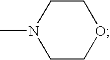
f. R₁ and R₂ together form a monocyclic ring of the formula 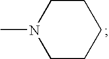
g. R₁ and R₂ together form a monocyclic ring of the formula

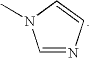

C₁-C₂₀Alkyl are straight-chain or branched alkyl radicals, for example methyl, ethyl, n-propyl, isopropyl, n-butyl, sec-butyl, tert-butyl, amyl, isoamyl or tert-amyl, heptyl, octyl, isooctyl, nonyl, decyl, undecyl, dodecyl, tetradecyl, pentadecyl, hexadecyl, heptadecyl, octadecyl or eicosyl.

C₃-C₈Cycloalkyl is, for example, cyclopropyl, cyclobutyl, cyclopentyl, cyclohexyl, cycloheptyl or cyclooctyl. Those radicals can be substituted, e.g. by one or more identical or different C₁-C₄alkyl radicals, especially by methyl, and/or by hydroxy. When cycloalkyl radicals are substituted by one or more substituents, they are preferably substituted by one, two or four, especially by one or two, identical or different substituents.

C₁-C₅Alkoxy are straight-chain or branched radicals, for example methoxy, ethoxy, propoxy, butoxy or pentyloxy.

Heteroaryl radicals can be unsubstituted or carry one or more, e.g. one, two, three or four, identical or different substituents, which may be located in any positions. Examples of such substituents are e.g. C₁-C₄alkyl, halogen, hydroxy, C₁-C₄alkoxy, trifluoromethyl, cyano, hydroxycarbonyl, C₁-C₄alkoxycarbonyl, aminocarbonyl, amino, C₁-C₄alkylamino, di-C₁-C₄-alkylamino and C₁-C₄alkylcarbonylamino.

Heteroaryl radicals are derived from heterocycles having one, two, three or four identical or different ring hetero atoms, especially from heterocycles having one, two or three, especially one or two, identical or different hetero atoms. The heterocycles can be mono- or poly-cyclic, e.g. mono-, bi- or tri-cyclic. They are preferably mono- or bi-cyclic, especially mono-cyclic. The rings preferably contain 5, 6 or 7 ring members. Examples of monocyclic and bicyclic heterocyclic systems from which radicals appearing in the compounds of formula (1) may be derived are, for example, pyrrole, furan, thiophene, imidazole, pyrazole, 1,2,3-triazole, 1,2,4-triazole, pyridine, pyridazine, pyrimidine, pyrazine, pyran, thiopyran, 1,4-dioxane, 1,2-oxazine, 1,3-oxazine, 1,4-oxazine, indole, benzothiophene, benzofuran, pyrrolidine, piperidine, piperazine, morpholine and thiomorpholine.

Unsaturated heterocycles can contain, for example, one, two or three unsaturated double bonds in the ring system. 5-Membered rings and 6-membered rings in monocyclic and polycyclic heterocycles may especially also be aromatic.

Preference is given to alkylbenzylamines of formula (1) wherein
R₁ is hydrogen; C₁-C₁₈alkyl; trifluoromethyl; C₃-C₈cycloalkyl; phenyl-C₁-C₅alkyl; phenyl-C₁-C₅alkoxy; mono- or di-N-C₁-C₅alkylamino-C₁-C₅alkyl; amino-di-N-C₁-C₅alkylamino-C₁-C₅alkyl; C₁-C₅alkoxy-C₁-C₅alkyl;
R₂ is C₅-C₂₀alkyl; hydroxy-C₁-C₂₀alkyl; phenyl; phenyl-C₁-C₅alkyl; phenyl-C₁-C₅alkoxy; mono- or di-N-C₁-C₅alkylamino-C₁-C₅alkyl; amino-di-N-C₁-C₅alkylamino-C₁-C₅alkyl; or heteroaryl-C₁-C₅alkyl; or
R₁ and R₂ together with the nitrogen atom bonding them form a 6- or 7-membered mono-cyclic heterocyclic aromatic ring.

Special preference is given to compounds of formula (1) wherein
R₁ is hydrogen; C₁-C₈alkyl; benzyl; or together with R₂ forms a 5- to 7-membered monocyclic heterocyclic ring; and more especially hydrogen.
R₂ in formula (1) is preferably C₂-C₁₂alkyl; phenyl-C₁-C₂alkyl; hydroxy-C₁-C₅alkyl; heteroaryl-C₁-C₂alkyl; or R₂ forms together with R₁ a 5- to 7-membered monocyclic heterocyclic ring.

Special preference is given to compounds of formula (1) wherein
R₂ is a branched C₃-C₈alkyl radical, especially an isopropyl; isobutyl, tert-butyl; isohexyl; or isooctyl radical.

Special preference is given to compounds of formula (1) wherein R₁ and R₂ have the same meanings.

Of those compounds, preference is given to those wherein
R₁ and R₂ are linear C₂-C₁₂alkyl; or benzyl.

Special preference is given to compounds of formula (1) wherein
R₁ is hydrogen; or methyl; and
R₂ is C₂-C₁₂alkyl; or phenyl-C₁-C₂alkyl,
   and more especially to compounds of formula (1) wherein
R₁ is hydrogen.

Very special preference is given to compounds of formula (1) wherein
R₁ is hydrogen; and
R₂ is octyl.

The preparation of the compounds according to the invention is carried out by processes known per se in accordance with the following scheme: 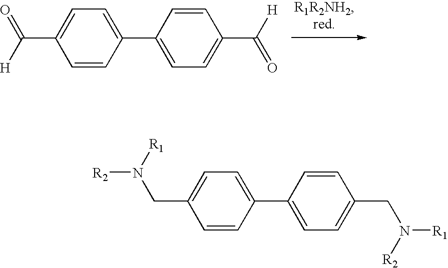
wherein R₁ and R₂ are as defined for formula (1).

The 4,4′-biphenyl carboxaldehyde is reacted with from 1 to 3 equivalents of amine and a reducing agent, e.g. hydrogen and a metal catalyst, formic acid, metal hydrides, e.g. borane complexes, borohydrides, aluminium hydrides, etc., in a suitable solvent, e.g. THF, DMF, dioxane, toluene, xylene, methanol or ethanol, with or without acid catalysis (acetic acid, TMOF) at a temperature of from −10° C. to 150° C. in the course of from 1 to 24 h, to form the corresponding amine compound.

In a further preparation variant, the bis-alkylbenzylamines according to the invention can be prepared in only one reaction step by direct alkylamination of BCMD. The process, which is a further subject of the invention, can be carried out as follows:

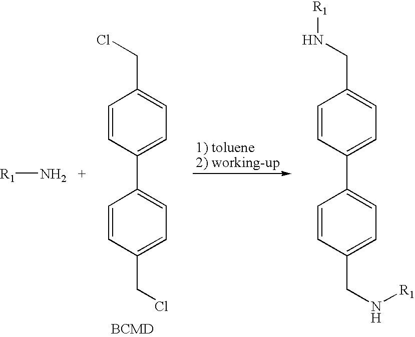

The alkylamination of BCMD is generally carried out in an excess of R₁—NH₂. The solvent (toluene) and the excess of R, —NH₂ can be recycled by distillation. The reaction time is from 0.5 to 12, preferably from 1 to 3 hours. The reaction temperature is from 50 to 120, preferably from 70 to 90° C.

Preferred solvents are toluene, xylene or fractions from petrol.

Examples of compounds according to the invention are listed in Table 1:

| Compound | | Purity |
|---|---|---|
| of formula | Structure | [254nm] |
| 2 | 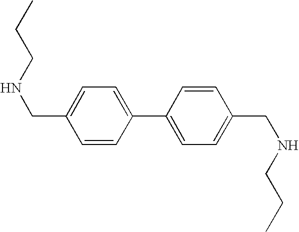 | 58 |
| 3 | 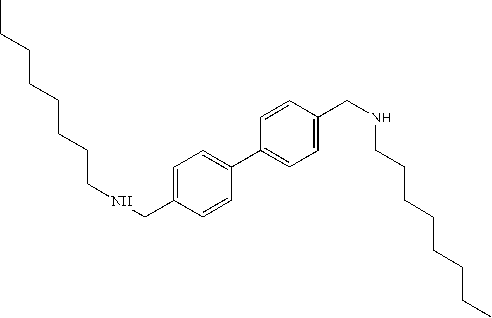 | 60 |
| 4 | 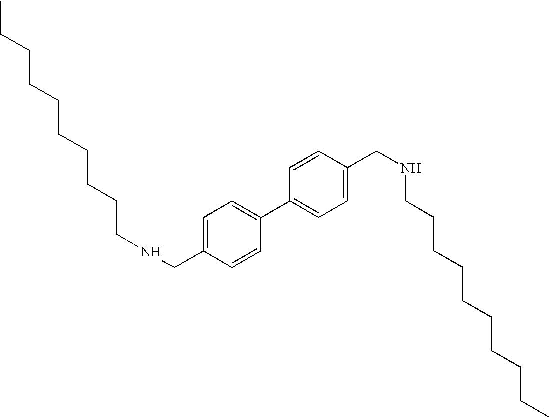 | 87 |
| 5 | 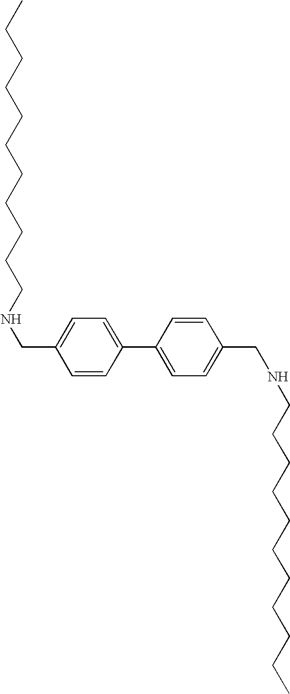 | 72 |
| 6 | 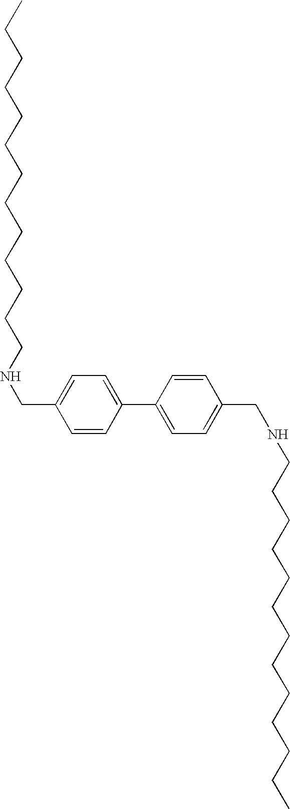 | 82 |
| 7 | 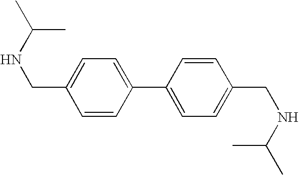 | 67 |
| 8 | 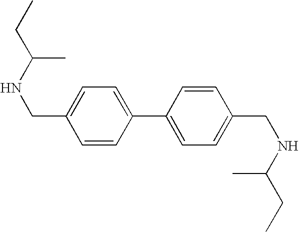 | 54 |
| 9 | 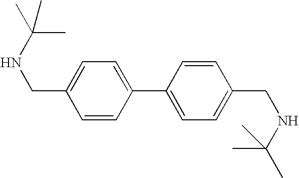 | 83 |
| 10 | 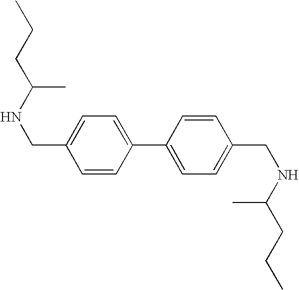 | 85 |
| 11 | 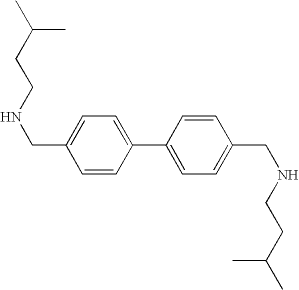 | 80 |
| 12 | 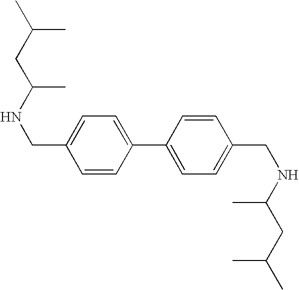 | 87 |
| 13 | 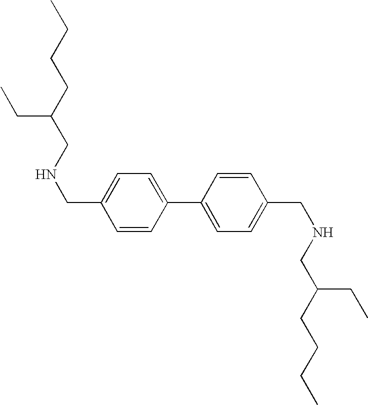 | 90 |
| 14 | 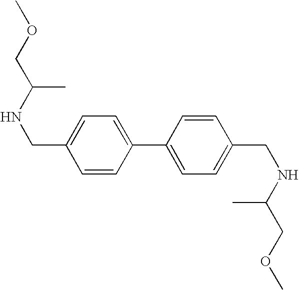 | 76 |
| 15 | 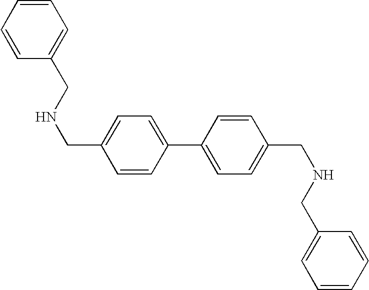 | 68 |
| 16 | 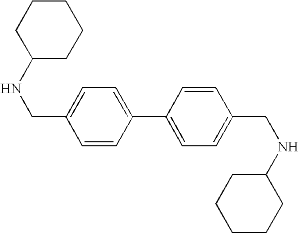 | 82 |
| 17 | 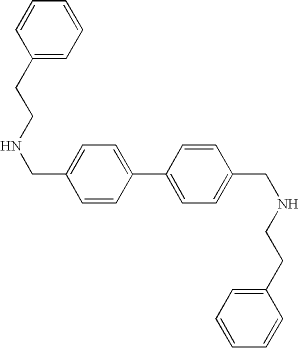 | 84 |
| 18 | 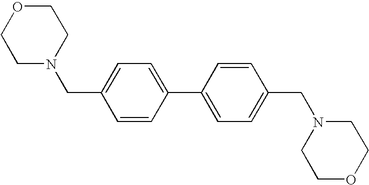 | 53 |
| 19 | 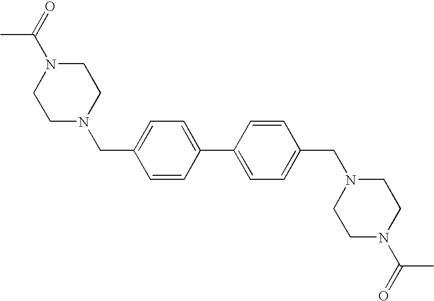 | 92 |
| 20 | 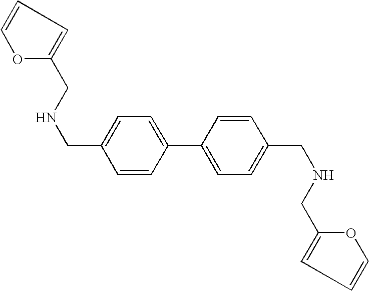 | 80 |
| 21 | 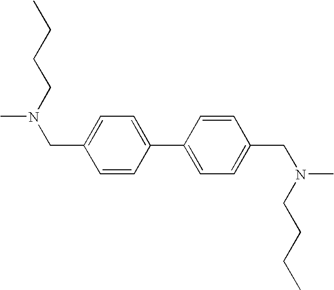 | 75 |
| 22 | 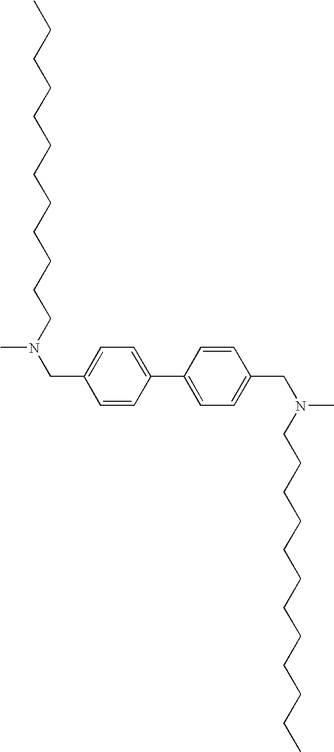 | 83 |
| 23 | 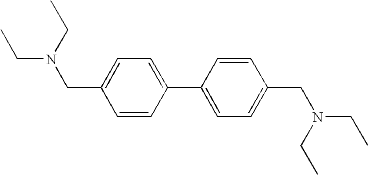 | 65 |
| 24 | 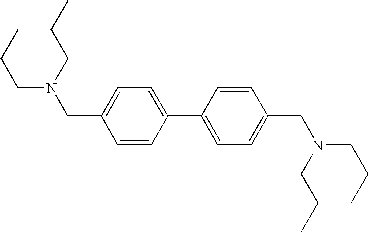 | 54 |
| 25 | 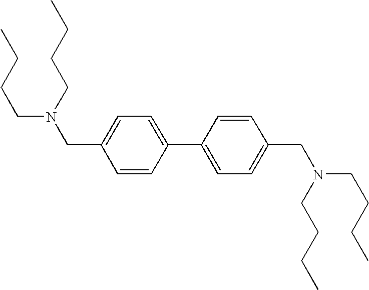 | 76 |
| 26 | 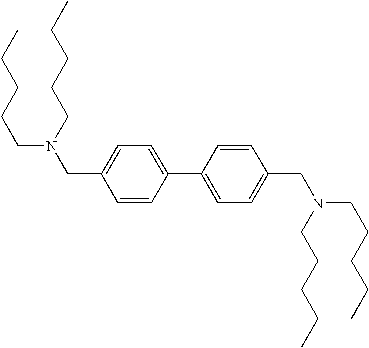 | 60 |
| 27 | 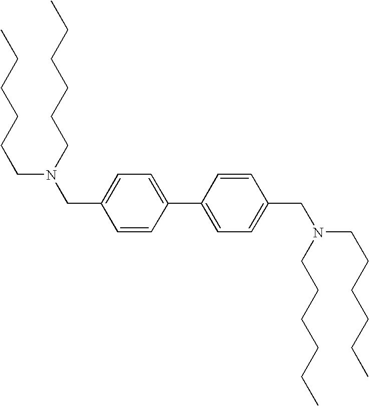 | 53 |
| 28 | 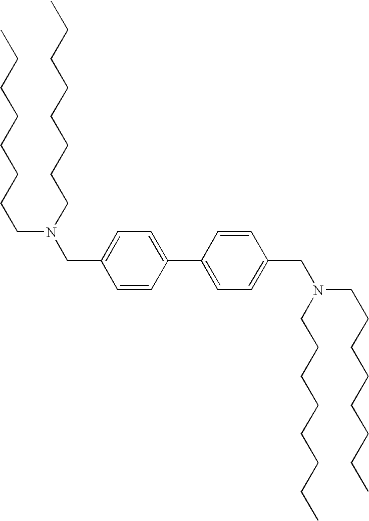 | 45 |
| 29 | 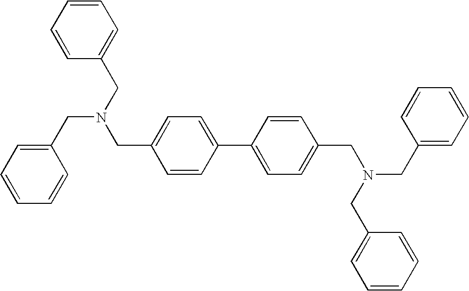 | 68 |
| 30 | 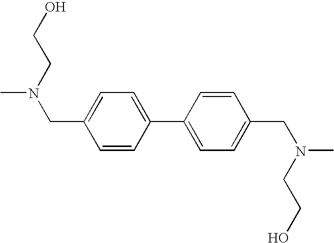 | 73 |
| 31 | 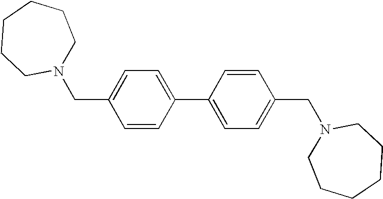 | 53 |

The bis-alkylbenzylamines used according to the invention exhibit a pronounced antimicrobial action, especially against pathogenic gram-positive and gram-negative bacteria and also against bacteria of skin flora. They are therefore especially suitable for the disinfection, deodorisation and the general and antimicrobial treatment of the skin and mucosa and also of integumentary appendages (hair), more especially for the disinfection of the hands and of wounds. They are therefore suitable as antimicrobial active ingredients and preservatives in personal care preparations, for example shampoos, bath additives, hair-care products, liquid and solid soaps (based on synthetic surfactants and salts of saturated and/or unsaturated fatty acids), lotions and creams, deodorants, other aqueous or alcoholic solutions, e.g. cleansing solutions for the skin, moist cleansing cloths, oils or powders.

The invention therefore relates also to a personal care preparation comprising at least one compound of formula (1) as well as cosmetically tolerable carriers or adjuvants.

The personal care preparation according to the invention comprises from 0.01 to 15% by weight, preferably from 0.1 to 10% by weight, based on the total weight of the composition, of bis-alkylbenzylamines of formula (1), and cosmetically tolerable adjuvants.

Depending upon the form of the personal care preparation, it comprises, in addition to the bis-alkylbenzylamine compound of formula (1), further constituents, for example sequestering agents, colourings, perfume oils, thickening or solidifying agents (consistency regulators), emollients, UV absorbers, skin-protective agents, antioxidants, additives that improve mechanical properties, such as dicarboxylic acids and/or Al, Zn, Ca and Mg salts of C₁₄-C₂₂-fatty acids, and optionally preservatives.

The personal care preparation according to the invention may be formulated as a water-in-oil or oil-in-water emulsion, as an alcoholic or alcohol-containing formulation, as a vesicular dispersion of an ionic or non-ionic amphiphilic lipid, as a gel, a solid stick or as an aerosol formulation.

As a water-in-oil or oil-in-water emulsion, the cosmetically tolerable adjuvant contains preferably from 5 to 50% of an oily phase, from 5 to 20% of an emulsifier and from 30 to 90% water. The oily phase may contain any oil suitable for cosmetic formulations, e.g. one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Cosmetic formulations according to the invention are used in a variety of fields. Especially the following preparations, for example, come into consideration:
skin-care preparations, e.g. skin-washing and cleansing preparations in the form of tablet-form or liquid soaps, synthetic detergents or washing pastes;
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, nail-care prep-arations, such as nail varnish, nail varnish removers, nail hardeners or cuticle removers;
intimate hygiene preparations, e.g. intimate washing lotions or intimate sprays;
foot-care preparations, e.g. foot baths, foot powders, foot creams or foot balsams, special deodorants and antiperspirants or callus-removing preparations;
light-protective preparations, such as sun milks, lotions, creams and oils, sun blocks or tropicals, pre-tanning preparations or after-sun preparations; skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks; deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or cream perfumes;
dental-care, denture-care and mouth-care preparations, e.g. toothpastes, gel tooth-pastes, tooth powders, mouthwash concentrates, anti-plaque mouthwashes, denture cleaners or denture fixatives;
cosmetic hair-treatment preparations, e.g. hair-washing preparations in the form of shampoos, hair conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidising dyes, or natural hair colorants, such as henna or camomile.

An antimicrobial soap has, for example, the following composition:

| | | |
|---|---|---|
| 0.01 to 5% | by weight | of a compound of formula (1) |
| 0.3 to 1% | by weight | titanium dioxide |
| 1 to 10% | by weight | stearic acid |

| | |
|---|---|
| ad 100% | soap base, e.g. the sodium salts of tallow fatty |
| | acid and coconut fatty acid or glycerol. |

A shampoo has, for example, the following composition:

| 0.01 to 5% | by weight | of a compound of formula (1) |
|---|---|---|
| 12.0% | by weight | sodium laureth-2-sulfate |
| 4.0% | by weight | cocamidopropyl betaine |
| 3.0% | by weight | NaCl and |

| | |
|---|---|
| ad 100% | water. |

A deodorant has, for example, the following composition:

| 0.01 to 5% | by weight | of a compound of formula (1) |
|---|---|---|
| 60% | by weight | ethanol |
| 0.3% | by weight | perfume oil and |

| | |
|---|---|
| ad 100% | water. |

The invention relates also to an oral composition, comprising from 0.01 to 15% by weight, based on the total weight of the composition, of a compound of formula (1), and orally tolerable adjuvants.

Example of an oral composition:

| | | |
|---|---|---|
| 10% | by weight | sorbitol |
| 10% | by weight | glycerol |
| 15% | by weight | ethanol |
| 15% | by weight | propylene glycol |
| 0.5% | by weight | sodium lauryl sulfate |
| 0.25% | by weight | sodium methylcocyl taurate |
| 0.25% | by weight | polyoxypropylene/polyoxyethylene block |
| | | copolymer |
| 0.10% | by weight | peppermint flavouring |
| 0.1 to 0.5% | by weight | of a compound of formula (1) and |
| 48.6% | by weight | water. |

The oral composition according to the invention may be, for example, in the form of a gel, a paste, a cream or an aqueous preparation (mouthwash).

The oral composition according to the invention may also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name Olafluor.

The bis-alkylbenzylamines of formula (1) according to the invention are also suitable for the treatment, especially the preservation, of textile fibre materials. Such materials are undyed and dyed or printed fibre materials, e.g. of silk, wool, polyamide or polyurethanes, and especially cellulosic fibre materials of all kinds. Such fibre materials are, for example, natural cellulose fibres, such as cotton, linen, jute and hemp, as well as cellulose and regenerated cellulose. Preferred suitable textile fibre materials are made of cotton.

The bis-alkylbenzylamines according to the invention are also suitable for the treatment of plastics, especially for imparting antimicrobial properties to or preserving plastics, e.g. polyethylene, polypropylene, polyurethane, polyester, polyamide, polycarbonate, latex etc. Fields of use therefor are, for example, floor coverings, plastics coatings, plastics container and packaging materials; kitchen and bathroom utensils (e.g. brushes, shower curtains; sponges, bathmats), latex, filter materials (air and water filters), plastics articles used in the field of medicine, e.g. dressing materials, syringes, catheters etc., so-called “medical devices”, gloves and mattresses.

Paper, for example papers used for hygiene purposes, may also be provided with anti-microbial properties using the bis-alkylbenzylamines according to the invention.

It is also possible for nonwovens, e.g. nappies/diapers, sanitary towels, panty liners, and cloths for hygiene and household uses, to be provided with antimicrobial properties according to the invention.

The bis-alkylbenzylamines of formula (1) are also used in washing and cleaning formulations, e.g. in liquid and powder washing agents or in softeners.

The bis-alkylbenzylamines can be used especially also in household and all-purpose cleaners for cleaning and disinfecting hard surfaces. A cleaning preparation has, for example, the following composition:

| | |
|---|---|
| 0.01 to 5% | of a compound of formula (1) |
| 3.0% | octyl alcohol 4EO |
| 1.3% | fatty alcohol C₈-C₁₀polyglucoside |
| 3.0% | isopropanol |
| ad 100% | water. |

Also possible, in addition to the preservation of cosmetic and household products, is the preservation of technical products and the provision of such products with antimicrobial properties as well as use as a biocide in technical processes, such as in paper treatment, especially in paper treatment liquors, printing thickeners of starch or of cellulose derivatives, surface-coatings and paints.

The bis-alkylbenzylamines of formula (1) are also suitable for the antimicrobial treatment of wood and for the antimicrobial treatment of leather, the antimicrobial preservation of leather and the provision of leather with antimicrobial properties.

The compounds according to the invention are also suitable for the protection of cosmetic products and household products from microbial spoilage.

The following Examples serve to illustrate the invention but do not limit the invention.

### EXAMPLES

### Example 1

### Preparation of octyl-(4′-octylaminomethyl-biphenyl-4-ylmethyl)-amine (Compound of Formula (3))

4.20 g (20 mmol) of 4,4′-biphenyl dicarboxaldehyde are dissolved in 50 ml of absolute THF under nitrogen. 4.80 g (80 mmol) of acetic acid and 5.69 g (44 mmol) of octylamine are added dropwise thereto and the mixture is heated for 1 hour at 60° C. After cooling with an ice-bath, 10.17 g (48 mmol) of sodium triacetoxyborohydride are added in portions. The reaction mixture is stirred overnight at room temperature. 100 ml of water are placed in a container and the reaction mixture is added using a pipette. The pH is adjusted to 1 with 6N hydrochloric acid. The THF is distilled off, and the product is filtered off and washed with 900 ml of water.

The product is then suspended in ethyl acetate, adjusted to pH 13 with sodium hydroxide solution and then filtered off again.

The product is washed with 300 ml of water. Excess octylamine is distilled off under a high vacuum.

Yield: 84% (GC purity: 88%)

NMR (methanol): 0.8 ppm (6H); 1.2 ppm (20H); 1.45 ppm (4H); 2.5 ppm (4H); 3.7 ppm (4H); 7.3 ppm (4H); 7.5 ppm (4H)

### Example 2

### Preparation of octyl-(4′-octylaminomethyl-biphenyl-4-ylmethyl)-amine (compound of formula (3))

A suspension of BCMD (1.00 mol) in toluene (2600 ml) is treated while cold with an excess of n-octylamine (5.00 mol). After reaction for one hour at 80° C., the conversion is complete. The reaction suspension is washed out with water (1000 ml) and 50% NaOH (200 g). The solvents (toluene/n-octylamine) are distilled off in vacuo (60-120° C./20 mbar).

The oily residue is taken up in warm ethyl acetate (1200 ml), clarified on a suction-filter (BCMD polymer), cooled to 35° C. and seeded.

After further cooling to 0° C., the crystalline product is filtered off, washed with solvent and dried.

### Example 3

### Determination of the Minimum Inhibiting Concentration (MIC Value) in Microtitre Plates

Nutrient Medium:

Casein/soybean flour peptone bouillon for the preparation of the precultures of the test bacteria and yeast.

Examples of Test Organisms:

Bacteria: Escherichia coli ATCC 10536 (=EC) Staphylococcus aureus ATCC 6538(=SA)

Procedure:

The test substances are predissolved in dimethyl sulfoxide (DMSO) and tested in a serial dilution of 1:2.

Bacteria and yeast are cultured overnight in CASO bouillon.

All test organism suspensions are adjusted to an organism count of 1-5×10⁶ CFU/ml with 0.85% sodium chloride solution.

The test substances are prepipetted into microtitre plates in an amount of 8 μl per well. The previously adjusted organism suspensions are diluted 1:100 in CASO bouillon and added to the test substances in an amount of 192 μl per well.

The test batches are incubated for 48 hours at 37° C.

After incubation, the growth is determined by reference to the turbidity of the test batches (optical density) at 620 nm in a microplate reader.

The minimum inhibiting concentration (MIC value) is the concentration of substance at which an appreciable inhibition of the growth (<20% growth compared with the growth control) of the test organisms is ascertained.

Three microtitre plates are used for each test organism and substance concentration.

The microbiological test results are compiled in Table 2:

TABLE 2

| | Purity | | |
|---|---|---|---|
| Compound of formula | [254 nm] | ATCC 6538 | ATCC 10536 |
| 2 | 58 | 15 | 15 |
| 3 | 60 | 7.5 | <3.75 |
| 4 | 87 | 30 | 15 |
| 5 | 72 | 30 | 30 |
| 6 | 82 | 30 | 30 |
| 7 | 67 | 15 | 30 |
| 8 | 54 | 15 | 60 |
| 9 | 83 | 60 | >120 |
| 10 | 85 | 15 | 30 |
| 11 | 80 | 30 | 30 |
| 12 | 87 | 30 | 60 |
| 13 | 90 | 7.5 | 7.5 |
| 14 | 76 | 30 | >120 |
| 15 | 68 | 120 | 120 |
| 16 | 82 | 60 | 120 |
| 17 | 84 | 15 | 15 |
| 18 | 53 | >120 | >120 |
| 19 | 92 | >120 | >120 |
| 20 | 80 | >120 | >120 |
| 21 | 75 | >120 | 120 |
| 22 | 83 | 15 | 15 |
| 23 | 65 | 15 | >120 |
| 24 | 54 | >120 | >120 |
| 25 | 76 | >120 | >120 |
| 26 | 60 | 30 | >120 |
| 27 | 53 | 15 | >120 |
| 28 | 45 | 7.5 | 15 |
| 29 | 68 | >120 | >120 |
| 30 | 73 | >120 | >120 |
| 31 | 53 | 60 | 120 |

### Example 4

### Determination of the Minimum Inhibiting Concentration of the Compound of Formula (3) in Respect of a Wider Spectrum of Organisms

The compound of formula (3) (see Table 1; GC purity 88%; prepared in accordance with Example 1) is tested on the microorganisms indicated in Table 3.

The test for determining the minimum inhibiting concentration is effected in an agar incorporation test.

TABLE 3

| Microorganism | | MIC |
|---|---|---|
| Staphylococcus aureus | ATCC 6538 | 3.75 |
| Staphylococcus aureus | ATCC 9144 | 3.75 |
| Staphylococcus epidermidis | ATCC 12228 | 3.75 |
| Corynebacterium xerosis | ATCC 373 | 1.88 |
| C. minutissimum | ATCC 23348 | 1.88 |
| Propionibacterium acnes | ATCC 6919 | 3.75 |
| Escherichia coli | NCTC 8196 | 3.75 |
| Escherichia coli | ATCC 10536 | 3.75 |
| Proteus vulgaris | ATCC 6896 | >120 |
| Klebsiella pneumoniae | ATCC 4352 | 7.5 |
| Salmonella choleraesuis | ATCC 9184 | 7.5 |
| Pseudomonas aeruginosa | ATCC 15442 | >120 |
| Candida albicans | ATCC 10231 | >120 |
| Aspergillus niger | ATCC 6275 | >120 |

1. An alkylbenzylamine of formula 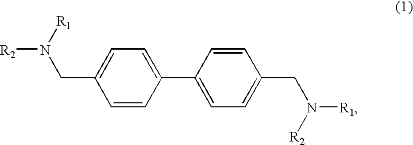
wherein
R₁ is hydrogen; C₁-C₁₈alkyl; trifluoromethyl; C₃-C₈cycloalkyl; phenyl-C₁-C₅alkyl; phenyl-C₁-C₅alkoxy; mono- or di-N-C₁-C₅alkylamino-C₁-C₅alkyl; amino-mono- or di-N-C₁-C₅alkylamino-C₁-C₅alkyl; or C₁-C₅alkoxy-C₁-C₅alkyl;
R₂ is C₂-C₂₀alkyl; hydroxy-C₁-C₂₀alkyl; phenyl; phenyl-C₁-C₅alkyl; phenyl-C₁-C₅alkoxy; mono- or di-N-C₁-C₅alkylamino-C₁-C₅alkyl; amino-mono- or -di-N-C₁-C₅alkylamino-C₁-C₅alkyl; or heteroaryl-C₁-C₅alkyl; or
R₁ and R₂ together with the nitrogen atom bonding them form a 5- to 7-membered monocyclic heterocyclic ring;
with the proviso that compounds of formula (1) are excluded wherein
a. R₁ is hydrogen; and
R₂ is butyl;
b. R₁ is hydrogen; and
R₂ is cyclohexyl;
c. R₁ and R₂ are butyl;
d. R₁ and R₂ are propyl;
e. R₁ and R₂ together form a monocyclic ring of the formula 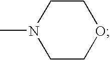
f. R₁ and R₂ together form a monocyclic ring of the formula 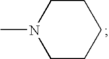
g. R₁ and R₂ together form a monocyclic ring of the formula

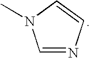

2. An alkylbenzylamine of formula 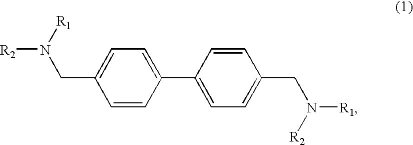
wherein
R₁ is hydrogen; C₁-C₁₈alkyl; trifluoromethyl; C₃-C₈cycloalkyl; phenyl-C₁-C₅alkyl; phenyl-C₁-C₅alkoxy; mono- or di-N-C₁-C₅alkylamino-C₁-C₅alkyl; amino-di-N-C₁-C₅alkylamino-C₁-C₅alkyl; or C₁-C₅alkoxy-C₁-C₅alkyl;
R₂ is C₅-C₂₀alkyl; hydroxy-C₁-C₂₀alkyl; phenyl; phenyl-C₁-C₅alkyl; phenyl-C₁-C₅alkoxy; mono- or di-N-C₁-C₅alkylamino-C₁-C₅alkyl; amino-di-N-C₁-C₅alkylamino-C₁-C₅alkyl; or heteroaryl-C₁-C₅alkyl; or
R₁ and R₂ together with the nitrogen atom bonding them form a 6- or 7-membered monocyclic heterocyclic aromatic ring.

3. A compound according to claim 1, wherein
R₁ is hydrogen; C₁-C₈alkyl; benzyl; or together with R₂ forms a 5- to 7-membered monocyclic heterocyclic ring.

4. A compound according to claim 1, wherein
R₁ is hydrogen.

5. A compound according to claim 1, wherein
R₂ is C₂-C₁₂alkyl; phenyl-C₁-C₂alkyl; hydroxy-C₁-C₅alkyl; heteroaryl-C₁-C₂alkyl; or together with R₁ forms a 5- to 7-membered monocyclic heterocyclic ring.

6. A compound according to claim 1, wherein
R₂ is a branched C₃-C₈alkyl radical.

7. A compound according to claim 6, wherein
R₂ is an isopropyl; isobutyl, tert-butyl; isohexyl; or isooctyl radical.

8. A compound according to claim 5, wherein
R₁ is hydrogen; and
R₂ is octyl.

9. A compound according to claim 1, wherein R₁ and R₂ have the same meanings.

10. A compound according to claim 9, wherein
R₁ and R₂ are linear C₂-C₁₂alkyl; or benzyl.

11. A compound according to claim 1, wherein
R₁ is hydrogen; or methyl; and
R₂ is C₂-C₁₂alkyl; or phenyl-C₁-C₂alkyl.

12. (canceled)

13. A method of antimicrobial treatment of a surface, which comprises contacting said surface with an antimicrobially effective amount of a compound of formula (1) wherein
R₁ is hydrogen; C₁-C₁₈alkyl; trifluoromethyl; C₃-C₈cycloalkyl; phenyl-C₁-C₅alkyl; phenyl-C₁-C₅alkoxy; mono- or di-N-C₁-C₅alkylamino-C₁-C₅alkyl; amino-di-N-C₁-C₅alkylamino-C₁-C₅alkyl; or C₁-C₅alkoxy-C₁-C₅alkyl;
R₂ is C₂-C₂₀alkyl; hydroxy-C₁-C₂₀alkyl; phenyl; phenyl-C₁-C₅alkyl; phenyl-C₁-C₅alkoxy; mono- or di-N-C₁-C₅alkylamino-C₁-C₅alkyl; amino-di-N-C₁-C₅alkylamino-C₁-C₅alkyl; or heteroaryl-C₁-C₅alkyl; or
R₁ and R₂ together with the nitrogen atom bonding them form a 5- to 7-membered monocyclic heterocyclic ring.

14. A method according to claim 13, wherein the compound is used in the deodorisation and disinfection of the skin, mucosa or hair.

15. A method according to claim 13, wherein the compound is used in the treatment of textile fibre materials.

16. A method according to claim 13, wherein the compound is used in the preservation and antimicrobial treatment of technical products.

17. A method according to claim 16, wherein the technical product is a plastic, paper, nonwovens, wood or leather.

18. A method according to claim 13, wherein the compound is used as an antimicrobial active ingredient in washing and cleaning formulations.

19. (canceled)

20. A personal care preparation, comprising
from 0.01 to 15% by weight, based on the total weight of the composition, of a compound of formula (1) according to claim 1, and cosmetically tolerable adjuvants.

21. An oral composition, comprising
from 0.01 to 15% by weight, based on the total weight of the composition, of a compound of formula (1) according to claim 1, and orally tolerable adjuvants.

22. A process for the preparation of a compound of formula (1) according to claim 1, wherein it is prepared in accordance with the following scheme: 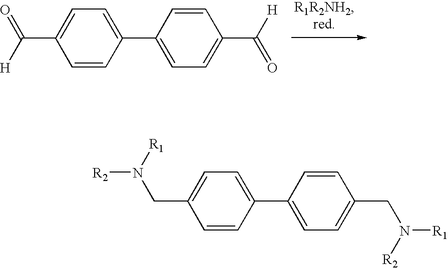
wherein R₁ and R₂ are as defined in claim 1.

23. A process for the preparation of a compound of formula (1) according to claim 1, wherein it is prepared in accordance with the following scheme: 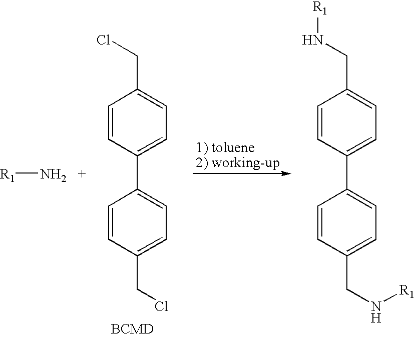
wherein R₁ is as defined in claim 1.
